(19) United States
(12) Patent Application Publication
(10) Pub. No.: US 2025/0128041 A1
(43) Pub. Date: Apr. 24, 2025

(54) ELONGATED TUBING FOR A ROTATING HEMOSTATIC VALVE

(71) Applicant: Contego Medical, Inc., Raleigh, NC (US)

(72) Inventors: Michael Hong, Oakland, CA (US); Aditya Priyonath Murugesh, Santa Clara, CA (US); Ajit Nair, Pleasanton, CA (US)

(21) Appl. No.: 18/920,210

(22) Filed: Oct. 18, 2024

Related U.S. Application Data
(60) Provisional application No. 63/591,707, filed on Oct. 19, 2023.

(51) Int. Cl. A61M 39/06; A61M 25/09; A61M 39/02

(52) U.S. Cl. CPC A61M 39/0613; A61M 25/09; A61M 2039/0258; A61M 2039/062; A61M 2039/0626; A61M 2039/0633

(57) ABSTRACT

An elongated tubing for use in a permissive seal assembly. The elongated tubing includes a proximal end, a distal end, and a widened central segment. A conformable path extends between the proximal and distal end, and a rigidified path extends between the proximal end and the distal end.

### CROSS REFERENCE TO RELATED APPLICATIONS

This application claims the benefit of U.S. Provisional Patent Application No. 63/591,707, filed on Oct. 19, 2023, the entire contents of which are incorporated herein by reference.

### FIELD

This disclosure relates to rotating hemostatic valves, and more particularly to rotating hemostatic valves intended to be used with guide wires, or rotating hemostatic valves intended to be used with devices fixed therewithin and a device movable therewithin.

### BACKGROUND

Introducer assemblies are used to aid the percutaneous introduction of cardiovascular catheters into a blood vessel. Introducer assemblies generally include an outer sheath (or an outer catheter) having a lumen extending therethrough. The outer sheath is inserted into an accessible vessel and stabilized therein, for example, by taping or suturing the sheath to the skin around the access site. Medical devices, guidewires, and/or smaller diameter catheters can then be inserted through the lumen of the outer sheath and into the vasculature and advanced to the desired position.

Hemostatic valves are typically positioned at the base, or hub, of an outer sheath to prevent unwanted blood loss, or unwanted air flow, through the lumen of the sheath. The valve mechanisms used in such device typically comprise one or more valve components formed of an elastomeric material with an opening to enable a catheter or some other instrument to be inserted therethrough. The elastomeric materials making up the prior art valve components typically exhibit a relatively high coefficient of friction. Thus, prior art introducers have tended to be somewhat resistant to the easy passage of a catheter or other instrument therethrough, hindering procedures that require repeated sliding of the catheter back and forth through the hub.

### SUMMARY

One implementation relates to an elongated tubing for use in a permissive seal assembly. The elongated tubing includes a proximal end, a distal end, and a widened central segment. At least one conformable path extends between the proximal and distal end, and a rigidified path extends between the proximal end and the distal end.

Another implementation relates to a permissive seal assembly that defines a fully open configuration and a partially closed configuration. The permissive seal assembly includes a rigid housing, a rigid compression mechanism, and a compressible elongated tubing that includes a widened central segment, at least one conformable path extending through the elongated tubing, and a rigidified path extending through the elongated tubing. The elongated tubing extends through a central channel defined by the compression mechanism and the housing. Radial compression of the compressible elongated tubing by the compression mechanism transitions the permissive seal assembly from the fully open configuration to the partially closed configuration, and movement from the fully open to the partially closed configuration substantially closes the conformable path but leaves the rigidified path substantially open.

Another implementation relates to a method of partially sealing a channel. The method includes passing a guidewire through a conformable path of an elongated tubing, the elongated tubing extending through the channel, passing a tool through a rigidified path of the elongated tubing, axially compressing a silicone gasket or conformable single-lumen tubing, translating axial compression to inward radial compression on the elongated tubing, substantially closing the conformable path to partially seal the channel, thereby restricting movement of the guidewire through the channel, and axially sliding the tool through the rigidified path while the conformable path remains substantially closed around the guidewire.

### DESCRIPTION OF DRAWINGS

The device is explained in even greater detail in the following drawings. The drawings are merely exemplary and certain features may be used singularly or in combination with other features. The drawings are not necessarily drawn to scale.

FIG. 1 is a side view of a thrombectomy system, according to some implementations.

FIG. 2 is a side view of an aspiration catheter hub used with the thrombectomy system of FIG. 1, according to some implementations.

FIG. 3 is a partial front view of a typical hemostatic valve, according to some implementations.

FIG. 4 is an exploded view of a rotating hemostatic valve of the aspiration catheter hub of FIG. 2, according to some implementations.

FIG. 5 is a photograph of the rotating hemostatic valve of FIG. 4 arranged in an open position, according to some implementations.

FIG. 6 is a photograph of the rotating hemostatic valve of FIG. 4 arranged in a partially closed position, according to some implementations.

FIG. 7 is a photograph of the rotating hemostatic valve of FIG. 4 arranged in a closed position, according to some implementations.

FIG. 8 is a photograph of the rotating hemostatic valve of FIG. 4 arranged in the closed position along with the thrombectomy system of FIG. 1 and a guide wire, according to some implementations.

### DETAILED DESCRIPTION

The following description of certain examples of the inventive concepts should not be used to limit the scope of the claims. Other examples, features, aspects, configurations, embodiments, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain advantages and novel features of the aspects and configurations of this disclosure are described herein. The described methods, systems, and apparatus should not be construed as limiting in any way. Instead, the present disclosure is directed toward all novel and nonobvious features and aspects of the various disclosed aspects, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Although the operations of exemplary aspects of the disclosed method may be described in a particular, sequential order for convenient presentation, it should be understood that disclosed aspects can encompass an order of operations other than the particular, sequential order disclosed. For example, operations described sequentially may in some cases be rearranged or performed concurrently. Further, descriptions and disclosures provided in association with one particular aspect or implementation are not limited to that aspect or implementation, and may be applied to any aspect or implementation disclosed. It will be understood that various changes and additional variations may be made and equivalents may be substituted for elements thereof without departing from the scope of the invention or the inventive concept thereof. Certain aspects and features of any given aspect may be translated to other aspects described herein. In addition, many modifications may be made to adapt a particular situation or device to the teachings of the invention without departing from the essential scope thereof. Therefore, it is intended that the invention not be limited to the particular implementations disclosed herein, but that the invention will include all implementations falling within the scope of the appended claims.

Features, integers, characteristics, compounds, chemical moieties, or groups described in conjunction with a particular aspect, configuration, implementation or example of the invention are to be understood to be applicable to any other aspect, configuration, embodiment, or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The claims are not restricted to the details of any foregoing aspects. The claimed concepts extend to any novel one, or any novel combination, of the features disclosed in this specification (including any accompanying claims, abstract, and drawings), or to any novel one, or any novel combination, of the steps of any method or process so disclosed.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. The terms “about” and “approximately” are defined as being “close to” as understood by one of ordinary skill in the art. In one non-limiting aspect the terms are defined to be within 10%. In another non-limiting aspect, the terms are defined to be within 5%. In still another non-limiting aspect, the terms are defined to be within 1%.

“Optional” or “optionally” means that the subsequently described event or circumstance may or may not occur, and that the description includes instances where said event or circumstance occurs and instances where it does not.

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

Certain terminology is used in the following description for convenience only and is not limiting. The words “right,” “left,” “lower,” and “upper” designate direction in the drawings to which reference is made. The words “inner” and “outer” refer to directions toward and away from, respectively, the geometric center of the described feature or device. The words “distal” and “proximal” refer to directions taken in context of the item described and, with regard to the instruments herein described, are typically based on the perspective of the practitioner using such instrument, with “proximal” indicating a position closer to the practitioner and “distal” indicating a position further from the practitioner. The terminology includes the above-listed words, derivatives thereof, and words of similar import.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

To continue discussion of the challenges described in the background section, hemostatic valves prevent blood loss and air entry through an access site during percutaneous procedures. However, hemostatic valves can also present challenges to the practitioner by inhibiting back-and-forth movement of a catheter or instrument through the hub of the outer sheath. Equipment used during a thrombectomy procedure will be used to illustrate the issue. However, it should be understood that this disclosure has broad applicability to all percutaneous procedures and should not be considered to be limited to thrombectomy procedures, or even to cardiovascular procedures.

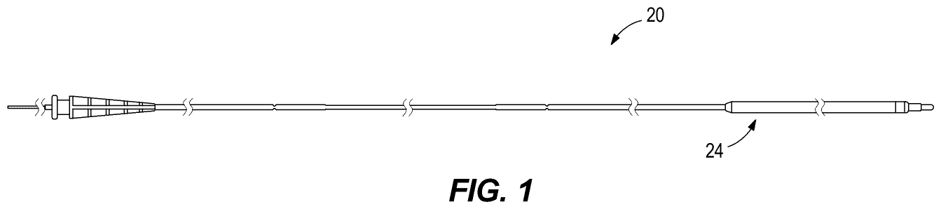

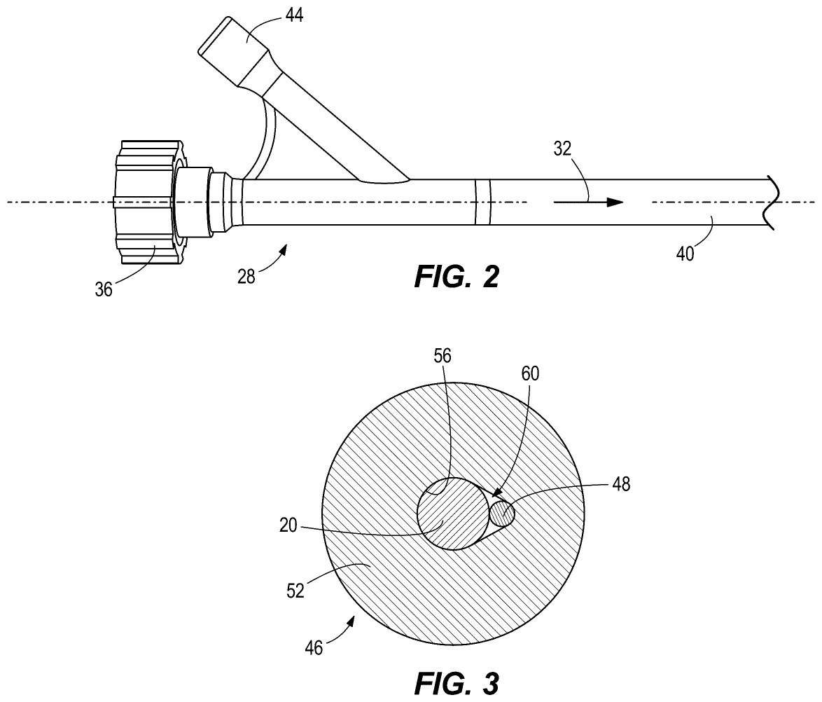

As shown in FIG. 1, a thrombectomy system 20 carries a thrombectomy retrieval device 24. As shown in FIG. 2, an aspiration catheter hub 28 is sized to receive a catheter of the thrombectomy system 20 in a direction illustrated by an arrow 32 into a permissive seal assembly in the form of a rotating hemostatic valve 36 of the aspiration catheter hub 28. During a thrombectomy procedure, the thrombectomy retrieval device 24 travels through an aspiration catheter 40, into the vasculature, and up to a thrombus, where it is expanded. The expanded retrieval device 24 scrapes the thrombus to dislodge pieces of it. The thrombectomy system 20 can then be retracted proximally by the practitioner. This proximal retraction causes the retrieval device 24 to carry pieces of the thrombus back to the distal opening of the aspiration catheter 40. The aspiration catheter 40, removes the thrombus pieces via an aspiration lumen 44, which is fluidically connected to a vacuum source. The thrombectomy system 20 can then be advanced distally again, pushing the retrieval device 24 back to the thrombus to scrape away more pieces.

During this procedure, it is important that the thrombectomy system 20 be able to easily move back and forth through the aspiration catheter hub 28 to reach the thrombus multiple times. However, it is also important to inhibit blood or air leaks through the rotating hemostatic valve 36. Furthermore, given the vacuum applied via aspiration lumen 44, it is important to seal the rotating hemostatic valve 36 from air flow because air leaks can disrupt the downstream aspiration systems, sensors, and/or algorithms designed to minimize blood loss that occurs by aspiration.

As shown in FIG. 3, a typical hemostatic valve 46 can be used in procedures that utilize the aspiration catheter hub 28 to stabilize a guidewire 48 while the thrombectomy system 20 moves back and forth directly adjacent to the guidewire 48. The typical hemostatic valve 46 includes an elastomeric gasket 52 with a single opening 56 through which both the guidewire 48 and the thrombectomy system 20 extend. The thrombectomy system 20 and the guidewire 48 are in contact at contact point 60. Therefore, it is difficult to move the thrombectomy system 20 without also moving the guidewire 48. It is also difficult to completely seal the typical hemostatic valve 46 when using a guidewire 48 directly adjacent to a thrombectomy system 20 because the (round) guidewire 48 contacts the (round) typical hemostatic valve 46 at a small contact point 60 and gaps are formed adjacent the contact point 60 that are not filled by the elastomeric gasket 52. These gaps provide common sites for leakage of air and/or blood.

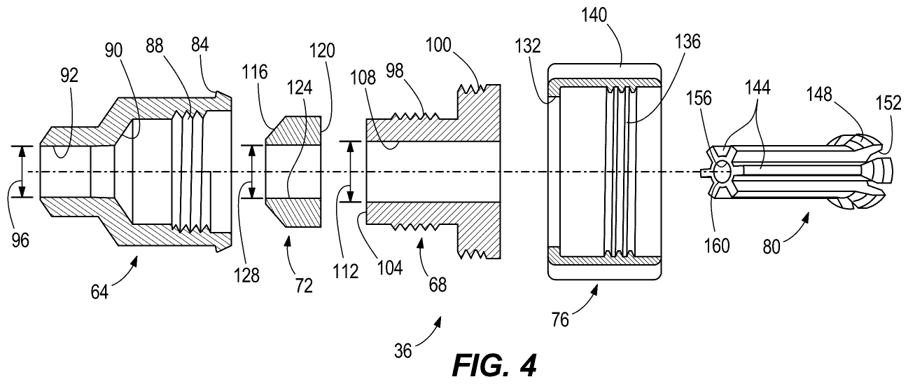

As shown in FIG. 4, the rotating hemostatic valve 36 of the aspiration catheter hub 28 includes a valve body 64, an inner knob 68 engaged with the valve body 64, a gasket 72 compressed between the valve body 64 and the inner knob 68, an outer knob 76 engaged with both the valve body 64 and the inner knob 68 to move the inner knob 68 relative to the valve body 64 and compress the gasket 72 therebetween, and a multi-passage insert 80 received through the gasket 72. The aspiration catheter hub 28 provides a hub for use with the thrombectomy system 20 and/or other catheter-based systems that inhibits leakage when used with a guidewire 48 or additional lumens/devices in concert with the thrombectomy system 20.

The valve body 64 includes a body flange 84 sized to engage the outer knob 76, a body thread 88 sized to receive the inner knob 68, a body sealing seat 90 sized to receive the gasket 72, and a body aperture 92 defining a body diameter 96 of 5.8 millimeters.

The inner knob 68 includes a first inner knob thread 98 sized to engage the body thread 88, a second inner knob thread 100 sized to engage the outer knob 76, an inner knob sealing surface 104 sized to engage the gasket 72, and an inner knob aperture 108 defining an inner knob diameter 112 of 6.9 millimeters. In some implementations, the inner knob 68 defines an inner knob length of 14.9 millimeters.

The gasket 72 is formed of an elastomer such as silicone or rubber and includes a first gasket sealing surface 116 shaped to seal against the seat 90, a second gasket sealing surface 120 shaped to seal against the inner knob sealing surface 104, and a gasket aperture 124 defining a gasket diameter 128 of 5.8 millimeters. In some implementations, the gasket 72 defines a gasket length of 8.0 millimeters. The gasket diameter 128 and the gasket length are defined while the gasket aperture 124 is undeformed.

The outer knob 76 includes an outer knob shoulder 132 sized to engage the body flange 84, an outer knob thread 136 sized to engage the second inner knob thread 100, and a gripping surface 140 that provides an easy to grip and manipulate knob.

The multi-passage insert 80 includes six insert apertures 144 extending along a length of the multi-passage insert 80, an enlarged insert head 148 defining enlarged insert apertures 152, an insert aperture 156 extending through the length of the multi-passage insert 80 and isolated from the six insert apertures 144, and a insert sleeve 160 received within the insert aperture 156. The six insert apertures 144 define a gear-like pattern. In some implementations, more than six or less than six insert apertures 144 are provided. In some implementations, the shape of the insert apertures 144 is different (e.g., squared, v-shaped, etc.). The length of the multi-passage insert 80 is greater than the summed lengths of the inner knob 68 and the gasket 72 so that the enlarged insert apertures 152 is visible and engageable by a user when the aspiration catheter hub 28 is assembled and in use. The enlarged insert apertures 152 provide access to the insert apertures 144 and are larger than the insert apertures 144 to improve case of access to the insert apertures 144. The enlarged insert head provide a ledge to inhibit the multi-passage insert from migrating into the hemostasis valve body or the aspiration catheter lumen. The insert sleeve 160 is optionally included and is formed of a lubricious material such as Polytetrafluoroethylene (PTFE) or PebaSLIX®. The multi-passage insert 80 is formed from a compressible material such as a rubber or Pebax® so that the insert aperture 156 constricts under compression.

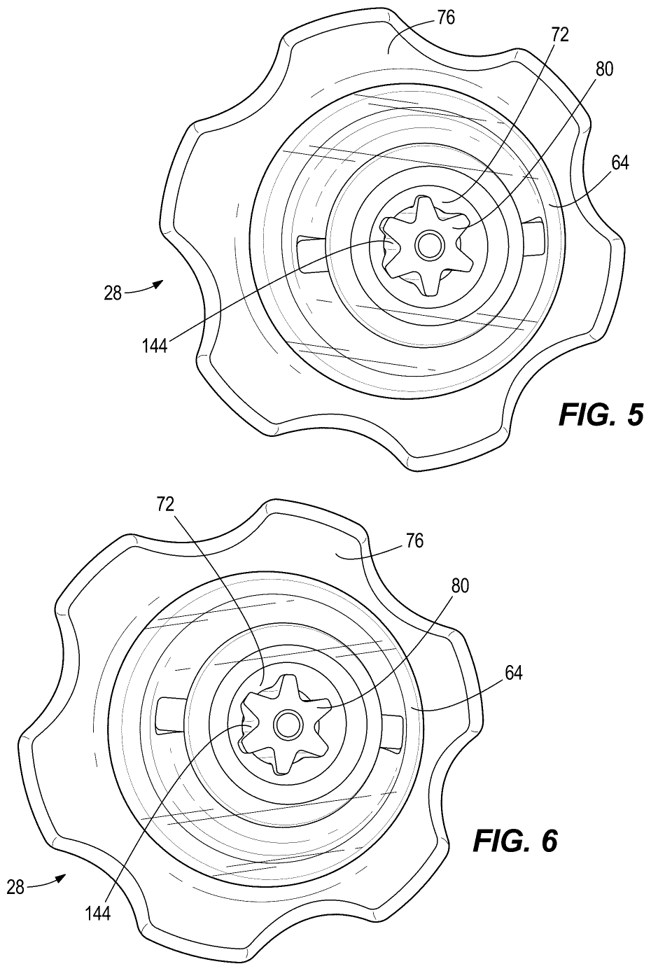

In operation, as shown in FIG. 5, the aspiration catheter hub 28 is arranged in an open position with the gasket 72 relatively uncompressed and space provided between the insert apertures 144 and the gasket 72. The open position allows insertion and manipulation of the guidewire 48 or other instruments inserted into the insert apertures 144.

As shown in FIG. 6, as the inner knob 68 is moved relative to the valve body 64, the gasket 72 is compressed therebetween and deforms into the insert apertures 144 in a partially closed position. The partially closed position defines less space within the insert apertures 144 than the open position.

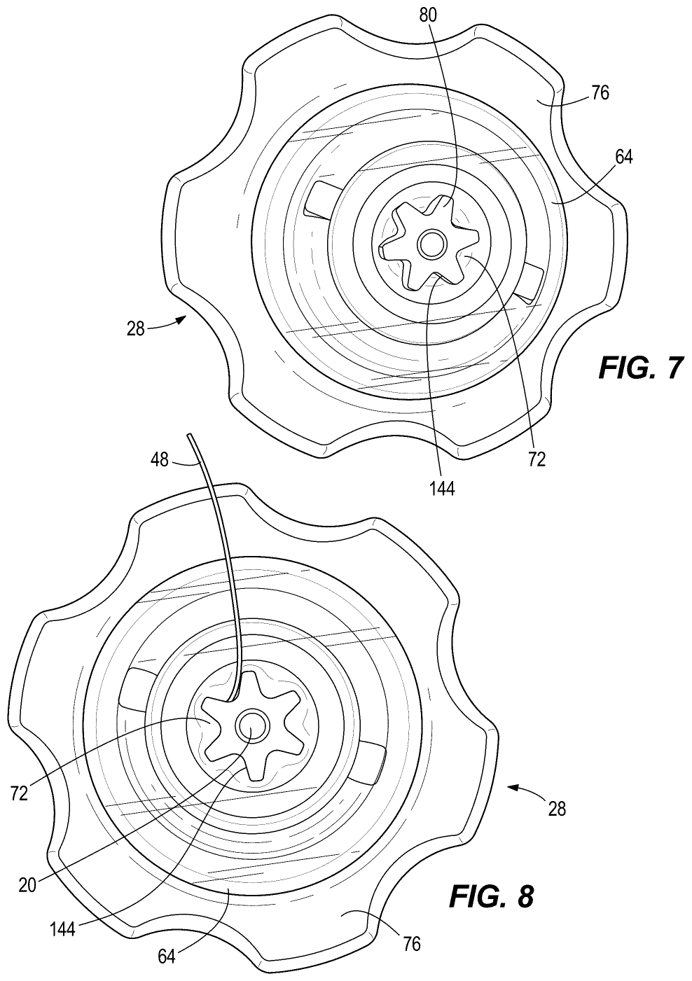

As shown in FIG. 7, continued tightening of the inner knob 68 relative to the valve body 64 further compressed the gasket 72 and provides a closed position wherein the insert apertures 144 are completely sealed with the gasket 72. In the closed position, leakage through the insert apertures 144 is inhibited. The shape of the insert apertures 144 allows for a smooth deformation of the gasket 72 as the aspiration catheter hub 28 is moved between the open position and the closed position so that effective sealing of the insert apertures 144 is possible. In the closed position, the multi-passage insert 80 is also compressed so that the insert aperture 156 is reduced in diameter. In this way, the insert apertures 144 and the inner diameter of the insert sleeve 160 are adjustable to control movement of the guidewire 48 and the thrombectomy system 20 relative to each other and relative to the aspiration catheter hub 28.

As shown in FIG. 8, the thrombectomy system 20 is sized to pass through the insert sleeve 160 while the guidewire 48 is positioned in one of the insert apertures 144 and sealed therein by the gasket 72. The isolation of the insert sleeve 160 from the insert apertures 144 provides the user the ability to move the thrombectomy system 20 within the insert sleeve 160 independent of interaction or impact of the guidewire 48. The insert sleeve 160 (or the lumen without the sleeve) is able to compress and seal around the thrombectomy system 20 and still be lubricious.

The surfaces of the seat 90, the inner knob aperture 108, the gasket 72, and the multi-passage insert 80 provide a high friction interface which constrains movement of the guidewire 48 when the aspiration catheter hub 28 is arranged in the closed position. The size and material of the insert sleeve 160 provide smooth movement of the thrombectomy system 20 therewithin. In some implementations, the insert sleeve 160 is sized for the specific thrombectomy system 20 being deployed. In some implementations, the multi-passage insert 80 may be provided as a kit of parts with multiple different insert sleeves 160 useable with different diameter thrombectomy systems 20. In some implementations, the insert sleeve 160 is deformable such that thrombectomy systems 20 of different diameters can be used with a single insert sleeve 160. In some implementations, the multi-passage insert 80 and the gasket 72 together provide a seal for use with the rotating hemostatic valve 36.

In some implementations, the aspiration catheter hub 28 provides a Touhy-style valve that is actuatable from fully open at 17 French to fully closed and leak-inhibited.

In some implementations, the multi-passage insert 80 and the gasket 72 may be formed as a single element (e.g., via comolding, overmolding, adhesive, mechanical connection, etc.). In some implementations, the multi-passage insert 80 and the gasket 72 may be provided preinstalled on the thrombectomy system 20. For example, in situations where the diameter of the thrombectomy system 20 is larger than the gasket diameter 128, the multi-passage insert 80 and the gasket 72 may be preinstalled so that the preinstalled multi-passage insert 80 and gasket 72 can be received within the other components of the rotating hemostatic valve 36 of the aspiration catheter hub 28. Alternatively, the gasket 72 and/or the multi-passage insert 80 may be provided with a slit so that the thrombectomy system 20 can be installed without inserting the entirety of the thrombectomy system 20 through the insert sleeve 160.

In some implementations, the components, assemblies, systems described above can include an elongated tubing (e.g., the combination of the gasket 72 and the multi-passage insert 80) for use in a permissive seal assembly (e.g., the rotating hemostatic valve 36). The elongated tubing includes a proximal end, a distal end, and a widened central segment (e.g., the gasket 72). A conformable path (e.g., the insert apertures 144 and their interaction with the gasket 72) extends between the proximal and distal end, and a rigidified path (e.g., the insert aperture 156 and/or the insert sleeve 160) extending between the proximal end and the distal end.

In some implementations, the elongated tubing is formed of compressible material (e.g., rubber, Pebax®, silicone, a combination of materials, etc.). In some implementations, the widened central segment is detachable from the elongated tubing (e.g., the gasket 72 is separate from the multi-passage insert 80). In some implementations, an axial length of the widened central segment (e.g., the gasket 72) is from 20% to 40% of the axial length of the elongated tubing (e.g., the multi-passage insert 80).

In some implementations, a diameter of the rigidified path (e.g., the insert aperture 156 and/or the insert sleeve 160) is from 35% to 55% of an outer diameter of the widened central segment (e.g., the gasket 72). In some implementations, a diameter of the rigidified path is 0.5 mm to 1.5 mm. In some implementations, an axial length of the rigidified path (e.g., the insert aperture 156 and/or the insert sleeve 160) is equal to the distance between the proximal end of the rotating hemostasis valve and the distal end of the silicone gasket. In some implementations, the axial length of the rigidified path is 8 mm to 25 mm. In some implementations, the rigidified path comprises an inner surface material (e.g., the material of the insert sleeve 160), the inner surface material having a higher durometer than a durometer of a material adjacent to the inner surface material (e.g., the material of the multi-passage insert 80). In some implementations, the rigidified path comprises a rigid inner surface liner (e.g., the insert sleeve 160) formed of the inner surface material. In some implementations, an inner surface of the rigidified path has a higher lubricity as compared to at least one other surface of the elongated tubing.

In some implementations, the elongated tubing further includes two or more conformable paths (e.g., the six insert apertures 144). In some implementations, the rigidified path extends through a center of the elongated tubing (e.g., the insert aperture 156) and the two or more conformable paths are circumferentially spaced around the rigidified path (e.g., the gear shaped insert apertures 144).

In some implementations, the elongated tubing further includes a widened proximal segment (e.g., the enlarged insert head 148) that serves as a back-stop to inhibit the elongated tubing from migrating into the aspiration catheter lumen.

In some implementations, a permissive seal assembly (e.g., the rotating hemostatic valve 36) defines a fully open configuration or position and a partially closed configuration or position. The permissive seal assembly includes a rigid housing (e.g., the valve body 64), a rigid compression mechanism (e.g., the inner knob 68 and the outer knob 76), and a compressible elongated tubing (e.g., the gasket 72 and the multi-passage insert 80) that includes a widened central segment (e.g., the gasket 72), a conformable path (e.g., the insert apertures 144 of the multi-passage insert 80) extending through the elongated tubing, and a rigidified path (the insert aperture 156 and/or the insert sleeve 160) extending through the elongated tubing. The elongated tubing extends through a central channel (e.g., the inner knob aperture 108) defined by the compression mechanism and the housing. Axial compression of the compressible elongated tubing (e.g., the gasket 72) by the compression mechanism (e.g., inner knob 68) transitions the permissive seal assembly from the fully open configuration to the partially closed configuration, and movement from the fully open configuration to the partially closed configuration substantially closes the conformable path (e.g., seals the insert apertures 144) but leaves the rigidified path substantially open (e.g., the thrombectomy system 20 can still be moved within the insert sleeve 160). In other words, the gasket 72 closes or seals the insert apertures 144 while the interior diameter of the insert sleeve 160 is compressed to a lesser degree that allows for movement of the thrombectomy system 20 within the insert sleeve 160. Either axial compression of the gasket causes radial compression of the insert 80 or the insert 80 can be radially compressed directly.

In some implementations, the compression mechanism (the inner knob 68) nests within the housing (e.g., the valve body 64). In some implementations, the compression mechanism comprises at least one set of threads (e.g., the body diameter 96 and/or the second inner knob thread 100). In some implementations, the widened central segment (e.g., the gasket 72) is shaped to fit snugly within an enclosed space bounded at one end by the compression mechanism (e.g., the inner knob sealing surface 104 of the inner knob 68) and at another end by the housing (e.g., the seat 90 of the valve body 64). In some implementations, the enclosed space is axially longer in the open configuration than in the partially closed configuration.

In some implementations, a widened proximal segment (e.g., the enlarged insert head 148) of the elongated tubing extends beyond a proximal end of the central channel (e.g., the inner knob aperture 108).

In some implementations, a method of partially sealing a channel (e.g., the inner knob aperture 108 and/or the body aperture 92) includes passing a guidewire (e.g., the guidewire 48) through a conformable path (e.g., the insert apertures 144) of an elongated tubing (e.g., the gasket 72 and the multi-passage insert 80). The elongated tubing extends through the channel. The method further includes passing a tool (e.g., the thrombectomy system 20) through a rigidified path (e.g., the insert aperture 156 and/or the insert sleeve 160) of the elongated tubing, axially compressing the gasket 72 (e.g., via movement of the inner knob 68 relative to the valve body 64), translating axial compression to inward radial compression on the elongated tubing 80, substantially closing the conformable path to partially seal the channel, thereby restricting movement of the guidewire through the channel, and axially sliding the tool through the rigidified path while the conformable path remains substantially closed around the guidewire.

In some implementations, axially compressing the gasket includes rotating a screw (e.g., the inner knob 68) of a compression mechanism (e.g., the inner knob 68 and the outer knob 76). In some implementations, translating axial compression to inward radial compression comprises axially compressing a widened central segment (e.g., the gasket 72) of the elongated tubing in an enclosed and shrinking space (e.g., the space between the seat 90 of the valve body 64 and the inner knob sealing surface 104 of the inner knob 68).

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The implementation was chosen and described in order to best explain the principles of the invention and the practical application, and to enable others of ordinary skill in the art to understand the invention for various implementations with various modifications as are suited to the particular use contemplated.

1. An elongated tubing for use in a permissive seal assembly, the elongated tubing comprising:
a proximal end, a distal end, and a widened central segment;
a conformable path extending between the proximal and distal end; and
a rigidified path extending between the proximal end and the distal end.

2. The elongated tubing of claim 1, wherein the elongated tubing is formed of compressible material.

3. The elongated tubing of claim 1, wherein the widened central segment is detachable from the elongated tubing.

4. The elongated tubing of claim 1, wherein an axial length of the widened central segment is from 20% to 40% of the axial length of the elongated tubing.

5. The elongated tubing of claim 1, wherein a diameter of the rigidified path is from 35% to 55% of an outer diameter of the widened central segment.

6. The elongated tubing of claim 1, wherein a diameter of the rigidified path is from 0.5 mm to 1.5 mm.

7. The elongated tubing of claim 1, wherein the rigidified path comprises an inner surface material, the inner surface material having a higher durometer than a durometer of a material adjacent to the inner surface material.

8. The elongated tubing of claim 7, wherein the rigidified path comprises a rigid inner surface liner formed of the inner surface material.

9. The elongated tubing of claim 1, wherein an inner surface of the rigidified path has a higher lubricity as compared to at least one other surface of the elongated tubing.

10. The elongated tubing of claim 1, further comprising two or more conformable paths.

11. The elongated tubing of claim 10, wherein the rigidified path extends through a center of the elongated tubing and the two or more conformable paths are circumferentially spaced around the rigidified path.

12. The elongated tubing of claim 1, further comprising a widened proximal segment.

13. A permissive seal assembly comprising a fully open configuration and a partially closed configuration:
a rigid housing;
a rigid compression mechanism; and
a compressible elongated tubing comprising a widened central segment, a conformable path extending through the elongated tubing, and a rigidified path extending through the elongated tubing;
wherein the elongated tubing extends through a central channel defined by the compression mechanism and the housing;
wherein radial compression of the compressible elongated tubing by the compression mechanism transitions the permissive seal assembly from the fully open configuration to the partially closed configuration; and
wherein movement from the fully open to the partially closed configuration substantially closes the conformable path but leaves the rigidified path substantially open.

14. The permissive seal assembly of claim 13, wherein the compression mechanism nests within the housing.

15. The permissive seal assembly of claim 13, wherein the compression mechanism comprises at least one set of threads.

16. The permissive seal assembly of claim 13, wherein the widened central segment is shaped to fit snugly within an enclosed space bounded at one end by the compression mechanism and at another end by the housing.

17. The permissive seal assembly of claim 16, wherein the enclosed space is axially longer in the open configuration than in the partially closed configuration.

18. The permissive seal assembly of claim 13, wherein a widened proximal segment of the elongated tubing extends beyond a proximal end of the central channel.

19. A method of partially sealing a channel, the method comprising:
passing a guidewire through a conformable path of an elongated tubing, the elongated tubing extending through the channel;
passing a tool through a rigidified path of the elongated tubing;
axially compressing a gasket or conformably single-lumen tubing;
translating axial compression of the gasket or conformably single-lumen tubing to inward radial compression on the elongated tubing;
substantially closing the conformable path to partially seal the channel, thereby restricting movement of the guidewire through the channel; and
axially sliding the tool through the rigidified path while the conformable path remains substantially closed around the guidewire.

20. The method of claim 19, wherein axially compressing the gasket or conformably single-lumen tubing comprises rotating a screw of a compression mechanism.

21. The method of claim 19, wherein translating axial compression to inward radial compression comprises axially compressing a widened central segment of the elongated tubing in an enclosed and shrinking space.
